(19) United States
(12) Patent Application Publication
(10) Pub. No.: US 2013/0338732 A1
(43) Pub. Date: Dec. 19, 2013

(54) DYNAMIC COMPLIANCE VOLTAGE FOR ENERGY EFFICIENT STIMULATION

(71) Applicants: Case Western Reserve University, Cleveland, OH (US); The Cleveland Clinic Foundation, Cleveland, OH (US)

(72) Inventors: Thomas J. Foutz, Shaker Heights, OH (US); James R. Buckett, Chagrin Falls, OH (US); Cameron C. McIntyre, Cleveland, OH (US)

(21) Appl. No.: 13/920,665

(22) Filed: Jun. 18, 2013

Related U.S. Application Data
(60) Provisional application No. 61/660,919, filed on Jun. 18, 2012.

(51) Int. Cl. A61N 1/378

(52) U.S. Cl. CPC A61N 1/378
USPC 607/59; 607/2; 607/72; 607/62

(57) ABSTRACT

An apparatus and method are disclosed for providing efficient stimulation. As an example, a switched mode power supply can be configured to generate a dynamic compliance voltage based on a stimulus waveform that can be non-rectangular. An output stimulation signal can be supplied to one or more outputs based on the compliance voltage.

### CROSS-REFERENCE TO RELATED APPLICATION

This application claims the benefit of U.S. Provisional Patent Application No. 61/660,919, filed Jun. 18, 2012 and entitled DYNAMIC COMPLIANCE VOLTAGE FOR ENERGY EFFICIENT STIMULATION, which is incorporated herein by reference in its entirety.

### TECHNICAL FIELD

This disclosure relates to an apparatus and method for energy efficient stimulation.

### BACKGROUND

Electrical stimulation achieves neuromodulation by controlling the release of neurotransmitters in specific parts of the nervous system through induction of action potentials. Electrical stimulation involves transduction of electrical current from the device to ionic current in the nervous tissue. Extracellular methods have been developed to pass current into the tissue, affecting the extracellular voltage potential of the neuronal membrane. These methods typically utilize an implanted neurostimulator to deliver the electrical current.

Implanted electrical neurostimulators draw power from a finite energy supply (e.g., a battery), requiring either frequent recharge cycles or surgical replacement upon full discharge. Accordingly, batteries for conventional electrical neurostimulators must be sufficiently large to meet existing power requirements, which typically results in increased volume for implantable systems.

### BRIEF DESCRIPTION OF THE DRAWINGS

FIG. 1 depicts an example of a block diagram of a stimulation system.

FIG. 2 depicts examples of waveform shapes.

FIG. 3 depicts an example of a stimulation waveform.

FIG. 4 depicts an example of a stimulation waveform.

FIG. 5 depicts an example of another stimulation system that can be implemented.

FIG. 6 depicts an example of an implantable pulse generator device.

FIG. 7 depicts an example of a method for providing energy efficient stimulation.

### DETAILED DESCRIPTION

This disclosure relates to an apparatus for energy efficient stimulation. The apparatus can utilize non-rectangular waveforms to further improve efficiency. In one example, a multi-phasic waveform is provided in which a compliance voltage is controlled dynamically during a cathodic portion of the waveform period. The compliance voltage can vary (e.g., continuously over time) according to the modulation of the stimulus waveform. By dynamically varying the compliance voltage in this manner, a stimulator (e.g., an implantable stimulator) can provide energy efficient delivery of electrical therapy. This disclosure builds on that disclosed in U.S. patent application Ser. No. 13/288,673, filed Nov. 3, 2011 and entitled APPARATUS FOR ENERGY EFFICIENT STIMULATION, which claims benefit of U.S. Prov. Patent Appn. Ser. Nos. 61/409,701, filed Nov. 3, 2010 and 61/515,066, filed Aug. 4, 2011, each of which is incorporated herein by reference in its entirety. A copy of U.S. patent application Ser. No. 13/288,673 is attached as Appendix A and forms an integral part of this application.

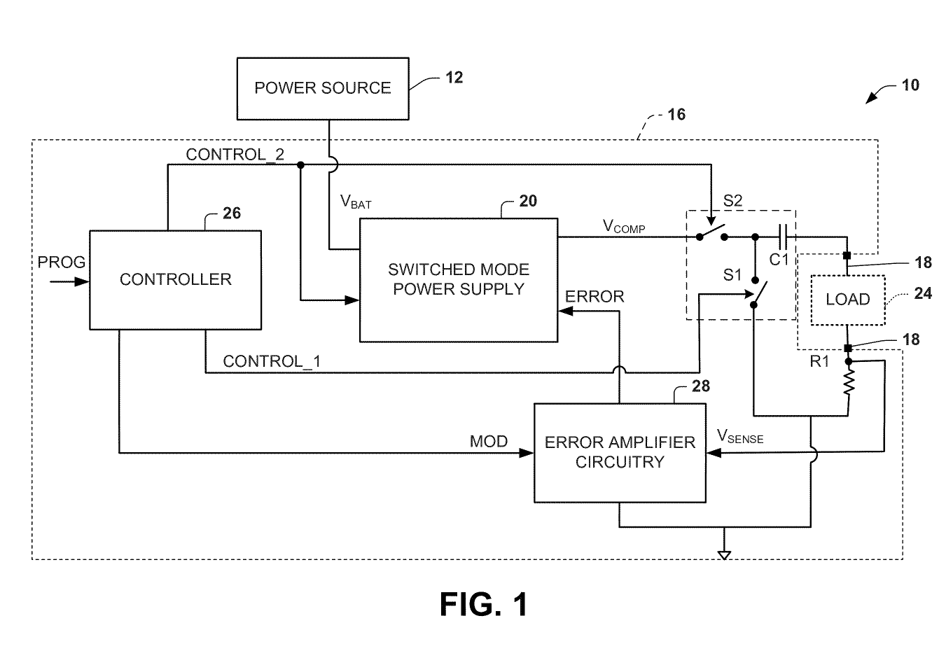

FIG. 1 illustrates an example of a block diagram of a system 10 for providing electrical stimulation. The system 10 includes a power source 12 that supplies power for the system 10. For example, the power source 12 can include one or more a battery that supplies a voltage V_{BAT} to a power input of a stimulation apparatus 16. The battery voltage V_{BAT} can be a DC voltage that depends on application requirements (e.g., ranging from 2.5 V to about 9 V DC).

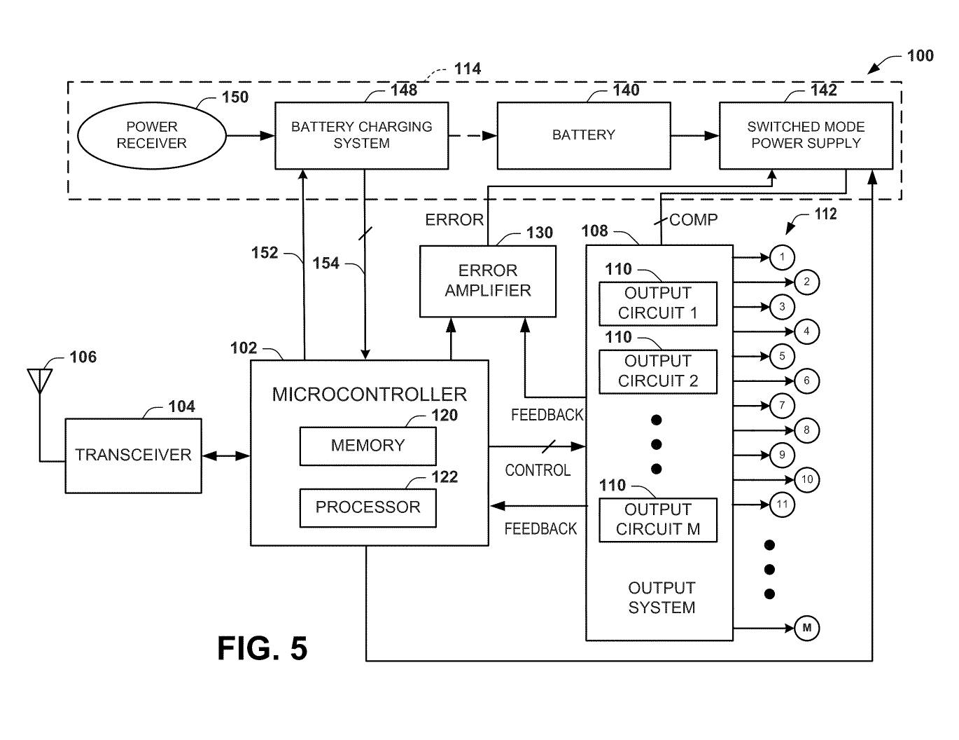

The stimulation apparatus 16 is configured to deliver an electrical therapy at one or more outputs 18 thereof. For simplicity of illustration, the example of FIG. 1 includes a single output; although there can be any number of output channels (see, e.g., FIG. 5). The system 10, including the power source and the stimulation apparatus 16, can be implemented as an implantable stimulation device in a self-contained housing that is hermetically sealed and capable of percutaneous implantation in a patient (e.g., human or other animal). As one example, circuitry in the stimulation apparatus 16 can be implemented as an application specific integrated circuit (ASIC).

The stimulation apparatus 16 can include an arrangement of circuitry that efficiently operates by dynamically managing power consumption during operation. In the example of FIG. 1, the apparatus 16 includes a switched mode power supply 20 that is connected to receive V_{BAT} from the power source 12. The switched mode power supply 20 can be configured to supply a compliance voltage V_{COMP} to output circuitry 22. The switched mode power supply 20 can dynamically vary the compliance voltage V_{COMP} according to an output waveform, demonstrated as MOD. The output circuitry 22 can be coupled to receive the compliance voltage V_{COMP} to supply corresponding electrical current to a load 24, which can be coupled to the output 18 of the stimulation apparatus 16.

As mentioned above, the stimulation apparatus 16 can include one or more outputs 18, each output corresponding to different output channel. Each output channel can be controlled independently of each other, such as to provide respective output waveforms to associated electrodes. That is each output 18 can be coupled to a corresponding load 24 that can include an electrode (not shown) and surrounding tissue (e.g., biological tissue at a target site in a patient's body) when implanted. There can be any number of electrodes and each such electrode can be dimensioned and configured according to application requirements. For example, the electrodes can be configured for delivery of electrical therapy to a target site or region in a patient, such as may be in the brain, spinal cord, peripheral nerves, vagus nerve, nerves for controlling bladder function, the heart or the like. In one example, the electrode can be formed of Iridium Oxide or include an Iridium Oxide coating.

The apparatus 16 can include a controller 26 configured to control operation of the apparatus. The controller 26 can be configured to control the output circuitry 22 to supply the stimulation signal to the load 24 based on the compliance voltage V_{COMP}. The output circuitry 22 can include an arrangement of switches S1 and S2 (e.g., transistor devices, such as field effect transistors (FETs)) configured to flow of electrical current based on control signals CONTROL_1 and CONTROL_2 provided by the controller 26 during respective phases of stimulation period.

For example, the controller can provide control signals CONTROL_1 and CONTROL_2 to close S2 and open S1 during a stimulus phase (e.g., a cathodic portion) of the stimulation period such that current is supplied to the load 24 via the output 18. During the stimulus phase, the current is supplied to the output 18 through a capacitor C1 based on the dynamic compliance voltage V_{COMP}. Thus, the capacitor C1 charges during the stimulus phase accordingly. The current through the load 24 also flows through a resistor R1. In the example of FIG. 1, R1 is connected between the load 24 and ground; although, in other examples, different configurations and placements of current sensors could be utilized. The current through the resistor R1 (corresponding to current through the load) provides a voltage potential V_{SENSE} across resistor R1 that can be provided as feedback to an input to error amplifier circuitry 28. The stimulus waveform MOD from the controller 26 can be provided as another input to error amplifier circuitry 28.

The error amplifier circuitry 28 can be configured to generate an ERROR signal based on the stimulus waveform MOD and the current through the load as indicated by the voltage V_{SENSE}. The resistor R1 and the error amplifier circuitry can be configured to normalize the stimulus waveform MOD and the voltage V_{SENSE} to a common scaled to enable a comparison to be performed. For instance, the error amplifier circuitry 28 can be configured to provide the ERROR signal based on a difference between the stimulus waveform and the sensed current to the load 24.

The switched mode power supply 20 can be configured to generate the compliance voltage V_{COMP} from the input battery voltage V_{BAT} based on the ERROR signal that is provided to the switched mode power supply 20. The control signal CONTROL_2 can also be provided to enable or disable the switched mode power supply. Thus, when the controller 26 provides the control signal CONTROL_2 to close the switch S2 (during the stimulus phase) the switched mode power supply 20 is concurrently activated to generate compliance voltage V_{COMP} based on the ERROR signal.

Following the stimulus phase, there may a small interphase interval before initiating the recovery phase of the stimulation period. During the recovery phase, the controller 26 can provide control signals CONTROL_1 and CONTROL_2 to open S2 and close S1 to discharge the capacitor C1 (or other energy storage element(s)). The capacitor C1 may maintain some charge but the total energy provided during each of the stimulus and recovery phases can be considered substantially charge-balanced. During the recovery phase the compliance voltage V_{COMP} can be fixed or it may be variable.

The controller 26 can be programmed to define parameters of the stimulus waveform such as based on a program (PROG) input. The stimulus waveform parameters for a given output can include amplitude, waveform shape, frequency (e.g., activation time), and pulse width (e.g., duration). The PROG input can be provided to the apparatus 16 via a physical connection (e.g., an electrical or optical link) or a wireless connection. Additionally or alternatively, other circuitry (not shown) in the system 10 can provide a portion of the PROG such as an indication of one or more sensed parameters (e.g., tissue impedance surrounding the electrode, electrophysiological signals sensed from the patient, feedback, such as voltage or current measurements, from circuitry in the system 10) that is utilized to dynamically adjust control signals and/or the stimulus waveform MOD. Regardless of changes in the stimulus waveform and related controls, the switched mode power supply 20 will supply a compliance voltage that varies dynamically during the stimulus phase based on the stimulus waveform.

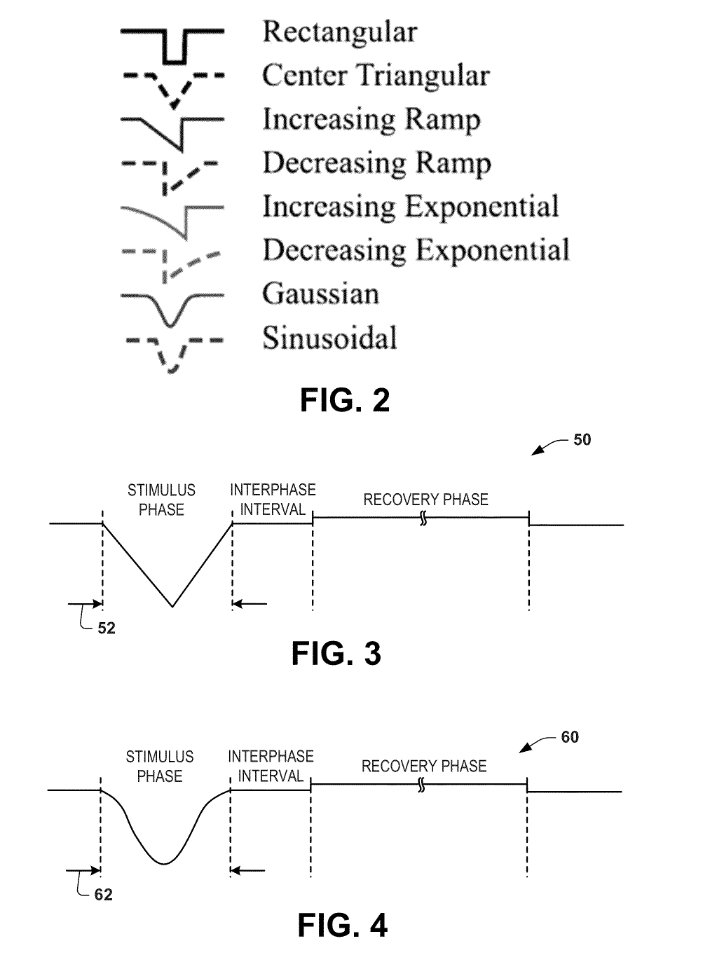

Examples of waveform shapes that can be provided by the controller 26 include rectangular, right triangular, centered triangular, increasing ramp, decreasing ramp, increasing exponential, decreasing exponential, Gaussian, sinusoidal, and trapezoidal to name a few. Examples of such waveform shapes are demonstrated in FIG. 2. In response to the stimulus waveforms from the controller 26, corresponding electrical current (or voltage) waveforms can be generated by the output circuitry 22 based on the compliance voltage. The waveforms may be analog waveforms or the shapes may be step-wise (e.g., discrete) approximations, which can vary depending on the capabilities of the controller 26 and application requirements. Examples of equations that can be used to generate various types of output current stimulus waveforms (I) are provided in the following table, where τ_{pw}, is the desired pulse width, I₀ is the amplitude, t is time and a and α are coefficients:

| Waveform Shape | Instantaneous Power |
|---|---|
| Rectangular: | I = I₀ |
| Sinusoidal: | I = I₀ sin(2πt/3τ_{pw}) |
| Centered triangular: | I = I₀ (1 − abs(t/τ_{pw} − 1)) |
| Right triangular: | $I = \frac{{I}_{0} t}{2 {τ}_{pw}}$ |
| Left triangular: | I = I₀ (1 − t/2τ_{pw}) |
| Gaussian: | $I = {I}_{0} \frac{exp ( at / {τ}_{pw} - {at}^{2} / {τ}_{pw}^{2} ) - 1}{exp ( a / 4 ) - 1}$ |
| Decreasing exponential decrease: | $I = {I}_{0} \frac{{e}^{α t / {τ}_{pw}} - 1}{{e}^{α} - 1}$ |
| Increasing exponential: | $I = {I}_{0} \frac{{e}^{α - α t / {τ}_{pw}} - 1}{{e}^{α} - 1}$ |

Energy and charge requirements can be determined for each waveform and pulse width using a corresponding threshold amplitude. The energy (E) of each cathodic stimulus of the waveform period can be ascertained by integration of the instantaneous power as follows:

E=∫₀^{T}^{ₑ}I²(t)Rdt
where T_{c} is the duration of the cathodic phase,
I(t) is the instantaneous current, and
R is the impedance (e.g., assumed constant: about 1 kΩ).
   As a further example, a passive anodic recharge phase is assumed. Consequently, the foregoing equation presumes to calculate stimulus energy only for the cathodic (stimulus) phase. The charge injected during stimulus can be determined (Q) by integrating the current over the cathodic (e.g., stimulus) phase (Tc):

Q=∫₀^{T}^{ₑ}I(t)dt

FIG. 3 demonstrates example of a stimulus waveform 50 demonstrating phases of a stimulation period. For example, the stimulation apparatus 16 of FIG. 1 can use charge-balanced, biphasic stimulus waveforms. As shown in the waveform 50, each stimulation period can be composed of three variable phases: a stimulus phase, an interphase interval, and a recovery phase. The stimulus phase can include a stimulus pulse that has an amplitude and pulse width 52, both of which can be programmed according to energy requirements and charge threshold levels, for example (e.g., by the PROG input to the controller 26 of FIG. 1). The example of FIG. 3 shows a centered triangle pulse during the stimulus phase. In other examples, other non-rectangular waveforms can be utilized with increased energy efficiency than rectangular waveforms. When considering non-rectangular waveform, the definition of pulse width becomes less clear, and can skew the interpretation of results. For purposes of consistency, the pulse width of each waveform can be represented by the full width at half maximum amplitude (FWHM). As a further example, waveforms can be constructed such that the interphase interval lasts 0.1 ms, followed by a 5.0 ms passive-recharge phase, which coincides with 136 Hz stimulation using modern DBS devices. Other phase timing can also be utilized.

As another example, FIG. 4 depicts another non-rectangular waveform 60, which is shown as a sinusoidal waveform. Similar to FIG. 3, the waveform 60 includes a stimulus phase, an interphase interval, and a recovery phase. In the example of FIG. 4, the waveform 60 includes a sinusoidal pulse during its stimulus phase 62.

As a further example, with reference back to FIG. 1, the input to the controller 26 can set stimulation parameters for a patient. For example, the input can select a type of waveform that is to be applied. The input can also define a pulse width for the stimulus waveform. Alternatively, or additionally, the controller 26 can determine the pulse width based on other information provided via the input. For example, the pulse width can be set as a function of the type of neuron, neuron anatomy (e.g., fiber diameter) and the type of waveform.

FIG. 4 depicts an example of an implantable pulse generator (IPG) system 100 that can be implemented. The IPG system 100 is configured to deliver electrical stimulation to target tissue via one or more output channels 112. In the example of FIG. 5, IPG the system 100 includes a microcontroller 102 that is operative to control application of stimulus pulses by providing control signals to respective output circuits 110 of an output system 108.

The output system 108 can include one or more such output circuits 110, demonstrated as including M circuits, where M is a positive integer. Each of the output circuits 110 can provide the output electrical signals (e.g., current pulses) to a set of one or more corresponding output channels 112 according to the control signals provided by the microcontroller 102 and based on a dynamic compliance voltage, such as disclosed herein. The output channels 112 may include output ports electrically coupled directly with respective electrodes or other peripheral devices coupled to receive the output waveforms from the IPG system 100. For example, the output circuits 110 can be configured to deliver electrical current at a desired level over a range from about 1 μA to about 20 mA.

In the example of FIG. 5, the microcontroller 102 can include memory 120 and a processor 122. The memory 120 can include data and instructions that are programmed to control operation of the IPG 100, such as may vary according to application design requirements of the IPG. The processor 122 can access the memory and execute the instructions stored therein. Alternatively, in other examples, the functionality of the microcontroller 102 could be implemented via a hardware design, such as configurable logic (e.g., a field programmable gate array (FPGA) or the like) that can be configured to function as disclosed herein. While the microcontroller 102 is demonstrated as an integrated unit, some of the functionality and related circuitry (e.g., sensors—not shown) that provide inputs to the microcontroller could be implemented as an external components implemented external to an integrated circuit comprising the microcontroller.

The microcontroller 102 can be coupled to a transceiver 104. The transceiver 104 can be coupled to an antenna 106 for implementing wireless communications to and from the IPG system 100. As used herein, the term “wireless” refers to communication of information without a physical connection for the communication medium (the physical connection usually being electrically conductive or optical fiber). As described herein, the transceiver 104 alternatively could be implemented as a hard wired connection (e.g., including electrically conductive and/or optical links). Those skilled in the art will understand and appreciate various types of wireless communication modes that can be implemented by the transceiver 104, such as described herein. As an example, the transceiver 104 can be programmed and/or configured to implement a short range bi-directional wireless communication technology, such as Bluetooth or one of the 802.11x protocols.

In addition to providing control signals to the output system 108, the microcontroller 102 is configured to provide one or more stimulus waveforms to an error amplifier 130. The error amplifier 130 also receives feedback from the output system 108. For example, the feedback can be a signal indicating current applied to a load by each active output circuit (e.g., from a current sense resistor connected in series with the load). When multiple output circuits 110 are activated concurrently, the system 100 can include plural error amplifiers (e.g., one for output circuit). The error amplifier 130 provides an error signal to a switched mode power supply 142.

As disclosed herein, the error signal represents a difference between the stimulus waveform and the actual stimulation signal (e.g., stimulation current) applied to the load by an activated output circuit. Since the modulation signal and the resulting error signal vary during the stimulus phase, the switched mode power supply 142 likewise dynamically varies the compliance voltage during the stimulus phase based on the error signal. The compliance voltage thus can define a dynamically varying voltage rail that is utilized by one or more output circuits 110 that are activated in a given stimulus phase of a stimulation period. The microcontroller 102 can also selectively activate and deactivate the switched mode power supply as to be active during the stimulus phase for providing the dynamic compliance voltage. It is understood that in some examples, there can be a separate switched mode power supply configured to supply a compliance voltage for each respective output circuit 110, each of which can operate independently based on control by the microcontroller 102. In other examples, a given switched mode power supply 142 can supply a dynamic compliance voltage to more than one output circuit 110.

The IPG system 100 can also include a power system 114 that includes the switched mode power supply 142 operative to supply a compliance voltage to a power rail for operation of IPG. Each of the output circuits 110 as well as other circuitry in the IPG 100 can be coupled to the power supply rail 144 corresponding to the compliance voltage. Additionally, the power system 114 can operate in multiple modes, such as including the fixed compliance mode, adjustable compliance mode and dynamic compliance mode disclosed herein. For instance, if a peak current delivery of the electrical therapy involves less voltage than available from a power supply, the variable compliance regulator is configured to provide a substantially fixed compliance voltage to the supply rail. While a single rail is shown, it will be understood that, depending on voltage requirements of the circuitry in the system 100, there can be more than one rail, each of which may be independently controlled to provide a regulated voltage that can be fixed, adjustable and/or may be dynamically varied as disclosed herein.

The microcontroller 102 can also control operation of the transceiver 104, such as through a corresponding interface. As an example, during a programming mode, the microcontroller 102 can receive and send information via the transceiver 104 for programming stimulation parameters for the IPG 100. Alternatively, some or all of the IPG operating parameters can be pre-programmed and stored in memory 120. The programmable operating parameters can include, for example, waveform type, amplitude, pulse width, frequency, as well as control the number of pulse trains that are supplied to the output system 108 for delivery of electrical therapy. The microcontroller 102 can be further programmed to modify such operating parameters during operation to provide a modified version of the waveform (e.g., the modifications being based on feedback to provide for closed loop operation or based on external user input via the transceiver).

The microcontroller 102 can also control which of the plurality of output channels 112 are provided with corresponding output stimulus waveforms. For example, the output system 108 thus can selectively distribute output waveforms to one or more of the output channels 112 based upon the control instructions that define how such distribution is to occur.

As described herein, one or more electrodes can be coupled to each of the corresponding output channels 112 for delivering corresponding electrical therapy based on the waveforms provided to the corresponding outputs by the respective output circuits 110. The size and the configuration of the output system 108 can vary according to the number of output channels. In this example, the energy available to the electrical components varies according to the compliance voltage that can be dynamically varied by the power system 114.

As a further example, the output circuits 110 can be implemented as voltage-to-current converters configured to provide electrical current to each output channel to which one or more electrodes can be connected. To mitigate interference and help electrically isolate the respective output channels 112, the output circuits 110 can include capacitors between the output system 108 and the corresponding ports of the output channels 112. The capacitors can block DC currents during stimulation as well as mitigate sustained delivery of DC current by discharging through the output circuits 110 during the recovery phase.

The power system 114 includes a battery 140 that stores a charge for providing corresponding DC voltage to the IPG system 100. For example, the battery 140 supplies the DC output voltage to the switched mode power supply 142, which provides the compliance voltage. The amount of voltage provided the battery 140 can vary according to the power requirements of the IPG system 100. The battery 140 can be rechargeable.

The power system 114 can also include load tracking and additional switch mode power supplies (not shown) for providing appropriate power to other various parts of the IPG system 100. As disclosed herein, the switched mode power supply 142 can be a DC-DC boost converter that dynamically varies the voltage rail 144 available to the output system 108 and other circuitry as a function of the particular stimulus waveform(s) being provided by the microcontroller 102 to the output system 108. For example, the microcontroller 102 can provide a control signal based on one or more of the stimulus waveform and other operating parameters in response to which the switched mode power supply 142 dynamically varies the compliance voltage.

The output system 108 can also provide feedback to the microcontroller 102. As one example, the feedback can provide an indication of the output impedance for the respective output channels (e.g., including the impedance of the electrodes connected at the respective output channels and/or the impedance at the tissue/electrode interface). The microcontroller 102 or other circuitry can determine the impedance, for example, as a function of a voltage or current signal corresponding to the feedback. For example, the feedback can be utilized to fine tune the compliance voltage to increase the energy efficiency of the IPG 100. For example, a variable resistance element can be connected in series with the output of each output channel, which can be dynamically adjusted by the microcontroller based on such feedback.

The microcontroller 102 can also employ the transceiver 104 for transmitting appropriate information when the feedback indicates these and other sensed conditions may reside outside of expected operating parameters. The microcontroller 102 can initiate transmission of the information automatically in response to detecting operation outside of expected operating parameters. Alternatively, the microcontroller 102 can store such information (e.g., in the memory 120) and transmit in response to being interrogated by a corresponding external transmitter or external transceiver.

The power system 114 can also include a battery charging system 148 and a power receiver 150. The battery charging system 148, for example, may include charging control circuitry for the battery 140 as well as a power converter (e.g., including a rectifier) that is operative to convert the power received by the power receiver 150 to an appropriate form and level to facilitate charging the battery 140. In this regard, the battery 140 can be a rechargeable type, such as a lithium battery, or nickel cadmium battery capable of extended use between charges. Alternatively, the battery 140 may be replaceable (e.g., surgically or otherwise).

The power receiver 150, for example, can be implemented as a inductive power pick-up such as including an inductive coil and other appropriate circuitry that can receive, filter and couple power (e.g., via mutual inductance) from a corresponding power transmitter that may be placed adjacent or in contact with the power receiver. The power receiver 150 and the battery charging system 148 can be implemented as an integrated system to facilitate charging the battery 140. Additionally, the microcontroller 102 can control the battery charging system 148 in response to the feedback. For example, the microcontroller 102 can provide corresponding control signals 152 to the battery charging system 148 through a corresponding interface. Additionally, the current and/or voltage associated with the charging of the battery (or other parameters associated with operation of the charging system) can be monitored by the microcontroller 102 via one or more corresponding analog inputs 154. The microcontroller 102 can control the battery charging process in response to the voltage and/or current characteristics associated with the charging process, as detected via the input 154.

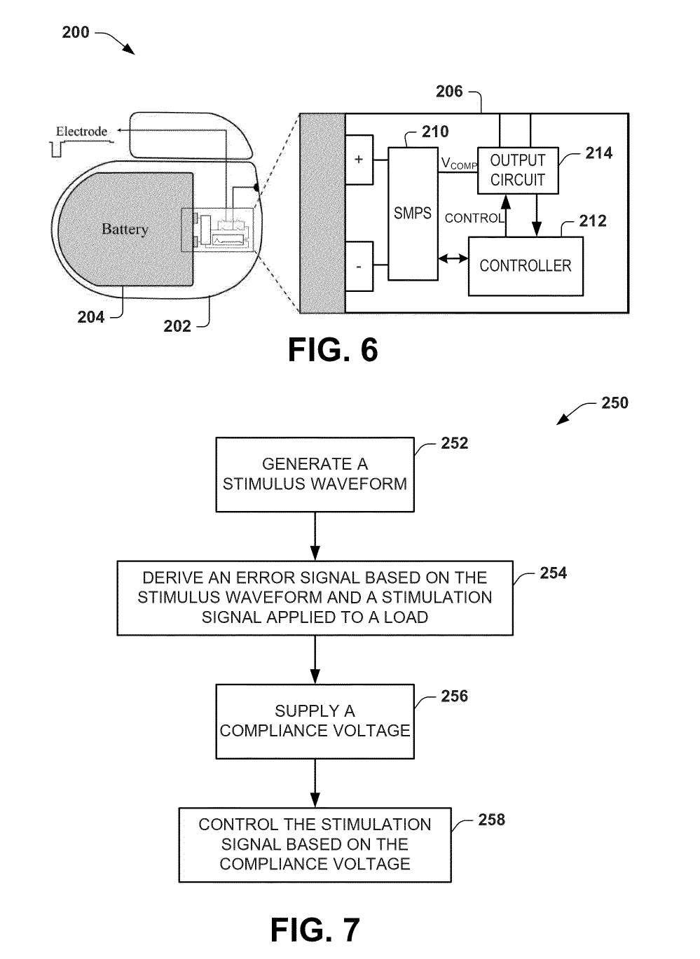

FIG. 5 depicts an example of an IPG device 200 as a self-contained unit (e.g., corresponding to the system 10 of FIG. 1 or system 100 of FIG. 5). The IPG device 200 includes a housing 202 that contains many components including a battery 204 and a stimulation apparatus 206. In the example of FIG. 6, the stimulation apparatus 206 includes a switched mode power supply 210, a controller 212 and an output circuit 214. For example, the cathodic stimulus waveform from the controller 212 can be utilized to generate a dynamic compliance voltage, which is utilized by the output circuit to generate a corresponding output current waveform based on the control signals from the controller, as disclosed herein.

Stimulator designs can vary depending on the device objectives, such as size, battery life, and application. In the case of DBS, the IPG can be implanted subcutaneously below the clavicle. The housing 202 can be hermetically sealed, and the IPG can be powered by a medical grade energy cell (battery 204). The IPG battery 204 can be rechargeable, which may require recharging daily or weekly. In other examples, an IPG may have a non-rechargeable battery, requiring surgical replacement every 3-6 years. The battery lifetime (or recharge interval) is dependent on the rate at which energy is consumed by each of the IPG circuit elements. Of all the neural stimulator's functions, stimulation is the largest energy consumer, and is therefore a primary target for increasing energy efficiency. Thus the approach disclosed herein for dynamically varying the compliance voltage for the IPG 100 can significantly increase energy efficiency.

In view of the foregoing structural and functional description, those skilled in the art will appreciate that portions of the invention (e.g., control functionality) may be embodied as a method, data processing system, or computer program product. Accordingly, these portions of the present invention may take the form of an entirely hardware embodiment, an entirely software embodiment, or an embodiment combining software and hardware. Furthermore, portions of the invention may be a computer program product on a computer-usable storage medium having computer readable program code on the medium. Any suitable computer-readable medium may be utilized including, but not limited to, static and dynamic storage devices, semiconductor storage devices, hard disks, optical storage devices, and magnetic storage devices.

In this regard, FIG. 7 depicts a method of performing stimulation of tissue, such as can be implemented with the systems 10, 100 or 200 disclosed herein. At 252, the method includes generating (e.g., by the controller 26 of FIG. 1) a stimulus waveform and a recovery waveform over each stimulation period. The stimulus waveform can be a non-rectangular modulated waveform such as disclosed herein. At 254, an error signal is derived (e.g., by error amplifier circuitry 28 of FIG. 1) based on a comparison between the stimulus waveform and a signal representing sensed stimulation signal that is applied to a load. At 256, the method includes supplying a compliance voltage that varies as a function of the error signal. The compliance voltage can be generated by a switched mode power supply (e.g., switched mode power supply 20 of FIG. 1) based on a control signal. At 258, an output circuit is controlled (e.g., by the controller 26 of FIG. 1) to generate the stimulation signal that is applied to the load according to the compliance voltage.

While the foregoing examples disclose dynamically varying the compliance voltage, corresponding to a voltage rail, which is used to supply an output waveform, similar effects can be achieved in a system that does not employ a compliance voltage per se. For example, a corresponding voltage rail can itself operate as the current driver for delivery of the output waveform to the load. In this implementation, the controller (e.g., the controller 26 of FIG. 1 or microcontroller 102 of FIG. 5) can adjust a corresponding voltage driver, which is coupled to provide an output voltage to the rail, based on the stimulus waveform. For instance, the controller can dynamically vary the voltage rail (via its control of the voltage driver) as to maintain the current through the load based on a sensed parameter (e.g., a feedback measurement from the load). In this way, a dynamic voltage pulse generator can be configured to provide a voltage output waveform, corresponding to a current stimulus waveform, where the voltage delivered by the stimulator is adjusted to maintain a constant load current, and the pulse generator voltage is adjusted dynamically (e.g., continuously in real time) in response to a current feedback measurement. The current feedback can be from the load itself or another element (e.g., a current sense resistor) in series with the load.

What have been described above are examples. It is, of course, not possible to describe every conceivable combination of components or methodologies, but one of ordinary skill in the art will recognize that many further combinations and permutations are possible. Accordingly, the invention is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

What is claimed is:

1. An apparatus, comprising:
a controller configured to provide a stimulus waveform and a control signal for different phases of stimulation period;
a switched mode power supply configured to supply a compliance voltage that varies as a function of the stimulus waveform and a sensed signal corresponding a stimulation signal supplied to drive a load;
an output circuit configured to supply the stimulation signal to drive the load based on the compliance voltage and the control signal.

2. The apparatus of claim 1, further comprising an error amplifier configured to generate an error signal based on a difference between the stimulus waveform and the sensed signal, the switched mode power supply configured to supply the compliance voltage based on the error signal.

3. The apparatus of claim 1, wherein the output waveform comprises a pulse having a pulse shape, the variable compliance regulator varies the compliance voltage dynamically to follow a pulse shape of the stimulus waveform for at least a cathodic portion of the stimulation period.

4. The apparatus of claim 1, wherein the output circuit includes a capacitor connected between the compliance voltage and the load, the output circuit being configured to discharge the capacitor during a recovery phase of the stimulation period.

5. The apparatus of claim 1, wherein the controller is programmed to supply the stimulus waveform with a pulse width according to at least one of a location of an electrode relative to a target, neuron type to be stimulated or waveform characteristics.

6. The apparatus of claim 1, wherein the stimulus waveform comprises at least one of a Gaussian waveform, right triangular waveform, centered triangular waveform, a rectangular waveform, a sinusoidal waveform, an increasing ramp waveform, a decreasing ramp waveform, an increasing exponential waveform and a decreasing exponential waveform.

7. The apparatus of claim 1, wherein the stimulus waveform comprises a centered triangular waveform during a stimulus phase and a rectangular waveform during a recovery phase of the stimulation period.

8. The apparatus of claim 1, wherein the controller dynamically controls the compliance voltage provided by the switched mode power supply based on a sensed parameter.

9. The apparatus of claim 8, wherein the sensed parameter corresponds to a signal sensed from circuitry residing in a stimulation path of the apparatus.

10. The apparatus of claim 8, wherein the sensed parameter corresponds to a characteristic of biological tissue at a target site.

11. The apparatus of claim 1, further comprising a power source coupled to provide a battery voltage to the switched mode power supply.

12. The apparatus of claim 1, wherein the controller is configured to control the switched mode power supply to operate in a mode selected from one of a dynamic operating mode, an adjustable operating mode and a fixed operating mode, the mode being selected based on a detected operating parameter.

13. An implantable pulse generation system comprising the apparatus claim 1, the system comprising an output system coupled to a supply rail, corresponding to the compliance voltage, the output system including at least one pulse generator to provide a corresponding output waveform to an associated output channel for delivering the stimulation signal.

14. A method comprising:
generating a stimulus waveform and a recovery waveform over each stimulation period;
deriving an error signal based on a comparison between the stimulus waveform and a signal representing a sensed stimulation signal that is applied to a load;
supplying a compliance voltage that varies as a function of the error signal; and
controlling an output circuit to generate the stimulation signal that is applied to the load according to the compliance voltage.

15. The method of claim 14, wherein the output waveform comprises a pulse having a pulse shape, the compliance voltage varying dynamically to follow a pulse shape of the stimulus waveform for at least a cathodic portion of the stimulation period.

16. The method of claim 14, wherein the output circuit includes a capacitor connected between the compliance voltage and the load, the controlling further comprising discharging capacitor during a recovery phase of each stimulation period.

17. The method of claim 14, further comprising programming a controller to supply the stimulus waveform with a pulse width according to at least one of a location of an electrode relative to a target, neuron type to be stimulated or waveform characteristics.

18. The method of claim 14, wherein the stimulus waveform comprises at least one of a Gaussian waveform, right triangular waveform, centered triangular waveform, a rectangular waveform, a sinusoidal waveform, an increasing ramp waveform, a decreasing ramp waveform, an increasing exponential waveform and a decreasing exponential waveform.

19. The method of claim 14, wherein the stimulus waveform comprises a centered triangular waveform during a stimulus phase and a rectangular waveform during a recovery phase of the stimulation period.

20. The method of claim 14, wherein the controlling further comprises dynamically controlling the supplying of the compliance voltage based on a sensed parameter.

21. The method of claim 20, wherein the sensed parameter corresponds to a sensed signal from circuitry in a stimulation path of the apparatus.

22. The method of claim 20, wherein the sensed parameter corresponds to a characteristic of biological tissue at a target site.

23. The method of claim 14, wherein the compliance voltage is provided by a switched mode power supply that is coupled to a batter, the method further comprising controlling the switched mode power supply to operate in a mode selected from one of a dynamic operating mode, an adjustable operating mode and a fixed operating mode, the mode being selected based on a detected operating parameter.
